(19) United States
(12) Patent Application Publication
(10) Pub. No.: US 2011/0250290 A1
(43) Pub. Date: Oct. 13, 2011

(54) DISINFECTANT COMPOSITIONS

(76) Inventors: Lyriam L. Marques, Edmonton (CA); Merle E. Olson, Calgary (CA); Michael W. Harding, Rosemary (CA)

(21) Appl. No.: 11/913,161

(22) Filed: Jan. 14, 2011

Related U.S. Application Data
(63) Continuation of application No. PCT/CA2007/001149, filed on Jun. 22, 2007.
(60) Provisional application No. 60/815,723, filed on Jun. 22, 2006.

(51) Int. Cl. A01N 59/16; A01P 1/00; C22C 5/06

(52) U.S. Cl. 424/618; 420/501

(57) ABSTRACT

The invention is a method and composition for treating biofilm using a silver deposition compound. In preferred embodiments of the invention, the anti-biofilm agent is used to preserve or disinfect plant material, including seeds, leaves, stems, vessels, flowers, roots and fruits.

### I. FIELD OF INVENTION

This invention relates to compositions and methods for treating plant materials. The compositions and methods also involve treating and/or preventing microbial contamination of plants, and to treating and preventing microbial plant diseases associated with biofilms. The compositions and methods may also be used as an anti-biofilm agent, more specifically an anti-microbial agent, effective against microorganisms including but not limited to bacteria, fungi, algae, viruses, and parasites. The compositions and methods may also be used as a disinfectant, and as a coating and/or ingredient in preventing or reducing hard surface contamination (e.g., paints or medical devices).

### II. BACKGROUND OF THE INVENTION

Recently, environmental, medical and industrial microbiologists have documented that microbial populations in their natural environments do not routinely grow as solitary or planktonic cells, but rather as biofilms; complex communities, attached to surfaces (Costerton et al., 1995; Davey and O'Toole, 2003). These discoveries have shifted the conceptual framework for treating a wide variety of microbiological diseases and conditions, including but not limited to plant pathology (Marques et al., 2002; Dow et al., 2002; Ramey et al., 2004); a wide variety of agricultural and farming applications; the food industry, particularly food processing surfaces; food borne illnesses, particularly Salmonella; food contamination and/or disease, including but not limited to Pierce's Disease in grapes, potato ring rot and storage rots, browning root rot, seed infestations; milk and milk products, a wide array of human and animal infections; medical implants; and medical devices.

Plant diseases cause world-wide economic losses in all industries involving plant production such as agriculture, horticulture, floriculture, turf-grass, nursery crops and forestry operations. In addition, pathogens attack plant materials in post-harvest storages. Global economic losses due to plant diseases were estimated at 10%-15% reduction in potential production resulting in a cost of $76.1 billion between 1988 and 1990 (Orke et al., 1994; Pinstrup-Anderson, 2001). These infections in plants and produce are caused predominantly by microorganisms such as fungi, bacteria, viruses, and nematodes.

Conventional commercial washing and sanitizing methods to remove microbial contaminants have been found to be marginally effective.

The presence of microbial and chemical pathogens at any stage of food production, processing, and distribution must be quickly determined in order to allow proper treatments before food consumption by the general public. There is a need for rapid, sensitive tests and effective intervention processes which researchers, farmers, processors, and retailers can use to verify that foods are safe to consume, and to reduce losses and operating costs.

As the horticultural market grows, it has become increasingly important to extend the life of plant material. Plant stresses can induce the production of ethylene (the “death” hormone), which accelerates senescence, affects flowering, fruit setting and ripening, seed germination, and defense against pathogens among other plant functions. (1) This combination of factors, along with increasing consumption of cut and potted flowers and the current level of international trade of flowers and fresh produce, has created a need for novel and effective approaches to preserve the freshness and quality of plant life.

Some commercial formulations that purport to extend the vase-life of flowers, usually include a sugar, which provides an energy source for the cut flowers and seems to protect against initial water loss (4), and may contain one or more microbial inhibitor(s) (5, 6, 7, 8).

Another major concern in plant production is the occurrence of seed bourne diseases. As an example, bacterial pathogens are a major problem in the production of dry bean (Phaseolus vulgaris) world-wide (Hirano and Upper, 1983; Singh and Munoz, 1999). Pathogens such as Pseudomonas syringae pv. syringae (brown spot), P. syringae pv. phaseolicola (halo blight), Xanthomonas axonopodis pv. phaseoli (common blight) and Curtobacterium flaccumfaciens pv. flaccumfaciens (wilt) cause serious losses in bean fields if the diseases are not managed. The use of certified disease-free seed is the first line of defense in preventing infections. Once diseases are introduced, the only method of control is the application of registered chemical pesticides. Foliar pesticides can reduce disease pressure, however chemical treatments applied to diseased fields must be applied repeatedly from the onset of symptoms until near harvest.

In contrast to this approach, disinfectant seed treatments provide a pre-emptive chemical treatment that aims to eradicate pathogenic bacteria and fungi from the seed surface, and those in the soil immediately adjacent to the seed, before and during germination and seedling development. (Coyne and Shuster, 1974; Finnisa and Tefera, 2001). Effective seed treatments can eliminate or delay the need for more costly applications of foliar pesticides.

A complicating factor in seed pathology is the ability of pathogenic bacteria to form biofilms, which are often highly resistant to removal and disinfection (Costerton et al. 1999; Ceri at al. 2001; Olson et al. 2002). As a result, past and current experimental results may dramatically overestimate the efficacy of chemicals used as anti-microbial cleaners, pesticides or disinfectants. It has been demonstrated that these four bacterial pathogens of dry bean can and do form biofilms either in vitro or in seeds (unpublished). In addition, these bacteria can form biofilms on seeds, resulting in current seed treatments being ineffective or marginally effective.

There is still a need for an efficacious, inexpensive anti-biofilm agent with the following properties: sustained release of anti-biofilm agent, ability to remove or degrade biofilms, and a low level of toxicity. This would be extremely beneficial to a very perishable commodity by lowering costs, increasing quality of plant material, increasing customer satisfaction, and promoting industry growth in the agricultural field as well as in the medical and industrial fields. Likewise, more effective and environmentally friendly seed treatments could help producers prevent plant diseases in greenhouse and field crops, reducing production losses and costly foliar pesticides applications later in the production cycle. Seed treatments can also help reduce the risk of seed-borne human infections such as those associated to sprouts.

### III. SUMMARY OF THE INVENTION

A need still exists for treating microorganisms with an anti-biofilm agent. The compositions and methods of the present invention have applicability in a wide variety of agricultural, industrial, and medical environments, e.g., extending or improving the life of plant material, particularly disinfecting seed surfaces; in anti-microbial coatings; and in treating human, plant, and animal diseases and conditions.

There is a need for a novel class of compounds with efficacy against microorganisms growing as biofilms

A need also still exists for an effective seed treatment in order to achieve improved disease management.

The invention provides a more efficient, practical, and environmentally friendly way to minimize crop losses and maximize post-harvest longevity and quality of plant agricultural and horticultural products, particularly seeds.

This invention also provides an effective method for the treatment of filamentous fungal biofilm.

### IV. DETAILED DESCRIPTION OF THE INVENTION

The present invention comprises compositions and methods for treating a biofilm using silver ions, preferably high valency silver. The compositions and methods may also include one or more other active agents. The compositions and methods are anti-microbial, including but not limited to bactericidal, fungicidal, viricidal, algicidal, or parasiticidal. As described in more detail below, the methods and compositions of the present invention may be used wherever biofilm or similar structures may be found, including but not limited to microorganisms growing and/or floating in liquid environments.

In some embodiments of the invention, the compositions and methods may be used to treat or prevent one or more biofilms, and/or to treat or prevent one of more human, animal, or plant diseases or conditions.

In some embodiments, the present invention comprises compositions and methods for preserving the health, life, or quality of plant material, including treating against bacteria, fungi, algae, biofilms, viruses, and parasites by contacting the plant material with a composition comprising one or more preservative agents. The preservative agent comprises a silver deposition product.

In some embodiments of the invention, the compositions and methods may be used to preserve and/or disinfect plants or parts thereof, most preferably seeds.

The invention also comprises contacting the plant material with a composition comprising one of more anti-biofilm agents, thereby extending storage life or preserving the plant material. In some embodiments of the invention, the anti-biofilm agent reduces or eliminates surface contamination.

In some embodiments of the invention, the compositions and methods may include applying the anti-biofilm agent to any portion of a plant, including but not limited to a cut or wounded surface of a plant; the roots, stems, leaves, or the seeds.

In accordance with some embodiments of the invention, any method of contacting the plant or portion thereof with an anti-biofilm agent may be used. Typical mechanisms for contacting the plant include but are not limited to coating, spraying, immersing, and diffusing in liquid, powder or other delivery forms.

Further, any harvesting or processing machinery surfaces, or tools can be impregnated with the anti-biofilm agent of the present invention.

Further, any storage, or greenhouse facilities or transport container can be impregnated with an anti-biofilm agent of the present invention so that the anti-biofilm agent prevents surface contamination and comes into contact with a plant or a portion thereof.

The compositions of the present invention may be used to treat a plant or portion thereof to eliminate or reduce one or more undesirable and/or deleterious microorganisms. The compositions of the present invention may be used to prevent one or more undesirable or deleterious microorganism from infecting a plant or portion thereof. In these embodiments of the invention, the preservative compositions and methods may be an anti-microbial agent.

The compositions of the present invention may be used to treat a plant or portion thereof to eliminate or reduce one or more undesirable and/or deleterious biofilms. The compositions of the present invention may be used to prevent one or more undesirable or deleterious biofilms from infecting a plant or portion thereof. In these embodiments of the invention, the preservative compositions and methods may be an anti-biofilm agent.

An embodiment of the invention comprises contacting plant material with an anti-biofilm agent composition comprising a metal or metal compound, preferably silver deposition compounds. One embodiment of the present invention comprises a composition comprising a silver deposition solution, and its use for preserving and extending the shelf life of plant material.

The invention may also include an apparatus and method for determining one or more anti-biofilm agents that are effective against one or more biofilms in plant material.

Silver deposition compositions, as used herein, refers to a electrochemical combination of a silver compound and Ag oxide(s). These compositions exhibit antimicrobial activity and/or anti-biofilm activity against both bacteria and fungi, and provide a sustained release of silver ions from silver compounds.

The silver containing deposition compositions produced using the methods of the invention may be comprised of silver ions having valent states higher than one, such as for example Ag (II) and Ag (III) valent states. It is also believed that silver containing deposition products produced using the methods of the invention may be comprised of silver ions having more than one valent state so that the oxidized silver species may be comprised of a multivalent substance. Finally, it is believed that silver containing deposition products produced using the methods of the invention may be comprised of a silver containing substance or a plurality of silver containing substances which react over time to form other silver containing substances which may exhibit differing antimicrobial properties. It is believed that if this is the case, the deposition products produced by the invention may be useful for providing a varied antimicrobial response and for overcoming bacterial resistance.

This invention demonstrates that stable, slow release silver deposition compounds, can be used as antimicrobials against bacteria and fungi pathogens, including biofilms, growing on plant surfaces or more broadly any hard surfaces associated with bacterial and fungal contaminants, i.e. dental implants, catheters.

In preferred embodiments of the invention, antimicrobial properties may be achieved by contacting a deposition product comprising an antimicrobially active silver species within or at the surface of a substrate, such as a plant material. These active silver species may include but are not limited to oxidized silver species such as silver salts, silver oxide (Ag₂O), higher silver oxides i.e. Ag(II) and Ag(III) (AgO, Ag₂O₃, Ag₃O₄ or like), silver oxy-salts with a general formula Ag₇O₈X where X can include one of acid anions such as sulfates, chlorides, phosphates, carbonates, citrates, tartrates, oxalates and like. The deposition product may also contain some elemental silver deposited during the process.

The term “oxidized silver species” as used herein may involve but is not limited to compounds of silver where said silver is in +I, +II or +III valent states or any combinations thereof. These oxidized silver species include, for example silver (I) oxide, silver (II) oxide, silver (III) oxide or mixtures thereof, all silver salts having a solubility product higher than 10-20 (such as for example Ag₂SO₄, AgCl, Ag₂S₂O₈, Ag₂SO₃, Ag₂S₂O₃, Ag₃PO₄, and the like), and silver oxy-salts such as Ag₇O₈X were X can include but is not limited at NO³⁻, ClO⁴⁻, SO₄²⁻, F— etc.

In the preferred embodiments, the deposition of the deposition product comprising the oxidized silver species may be accomplished by first providing an aqueous solution of monovalent silver salt or a silver complex such as silver nitrate, perchlorate or silver diamino complex, with silver nitrate being the most preferable if the reaction is carried out under acidic conditions or at close to neutral conditions (i.e. at pH below 7), and with silver diamino complex, (i.e., [Ag(NH₃)²]⁺) being the most preferable if the reaction is carried out under alkaline conditions (i.e. at pH above 7).

Preferably the deposition solution is agitated during at least a portion of the deposition product producing step in order to enhance the production of the deposition product.

The method according to a second aspect may further comprise immersing the composite material in boiling water, following the substrate contacting step. The immersing step may be useful for converting the deposition product into other oxidized silver species (such as silver oxides), thus potentially providing an opportunity further to “engineer” the composite material to provide desired properties of the deposition product. The immersing step may be performed for any length of time, but preferably the immersing step is performed for at least about 1 minute.

The silver deposition compounds may be used in any of the following formats: silver deposition coatings, e.g. on medical grade substrates or surfaces, liquid, powder, capsule, tablet, coating, and similar configurations, including but not limited to dressings, fibers, and materials composed of for example polyethylene, high density polyethylene, polyvinylchloride, polycarbonate, polyvynil-pyrrolidone (PVP), latex, silicone, cotton, rayon, polyester, nylon, wood, aluminum, steel, cement, cellulose, acetate, carboxymethylcellulose, alginate, chitin, chitosan, and hydrofibers.

A preferred embodiment of the present invention comprises a hydratable matrix material that has an antimicrobial agent, such as a heavy metal, most preferably, silver, incorporated into the matrix. The matrix preferably also comprises components that stabilize and control the release of the active agent into the surrounding environment when used.

In a preferred embodiment of the present invention, active agents are incorporated directly, or may be incorporated by sequentially adding components or precursors of the active agent to the matrix of the devices, and having the precursors of the active agent in or on the matrix. The agents may be incorporated by absorption or adsorption of agents or precursors by the matrix, and preferably by incorporation during the polymerization of the matrix. It is theorized that the release of the active agents may be controlled via manipulation of concentration parameters, movement of water through the matrix and the degree of cross-linking in the matrix. In another preferred embodiment, the wound dressings comprise a stranded configuration, wherein the strands extend from at least one common region and the strands themselves comprise a polymer matrix.

Other forms of the matrices of the present invention, such as molded articles, are also contemplated by the present invention. Other forms also include films, sheets, fibers and amorphous gels. The matrices of the present invention can be dipped or applied in methods known to those skilled in the art to articles or devices.

The present invention may also include the incorporation and stabilization of silver onto and within the hydrophilic fibers of cross-linked and non-cross-linked celluloses such as carboxymethyl cellulose and hydroxymethyl cellulose, cotton, rayon, and of fibers made from polyacrylates and other synthetic and natural polymers, and fibers of calcium alginates that may be used as a primary contact sustained-release silver antimicrobial materials.

Preferably, the hydrophilic matrix material is constructed from a natural or synthetic polymer and a non-gellable polysaccharide. Natural hydrophilic polymers that may be used include, but are not limited to collagen, arnal hide, hyaluronic acid, dextran and alginate. Additionally included are hydrophilic fibers of cross-linked and non-cross-linked celluloses such as carboxymethyl cellulose and hydroxymethyl cellulose; cotton, rayon, and of fibers made from polyacrylates; and fibers of calcium alginates that may be used as a primary contact sustained release silver antimicrobial material. Synthetic polymers that may be used include, but are not limited to polyacrylamide, polyvinyl's (PVP, and PVC), polyacrylate, polybuterate, polyurethane foam, silicone elastomer, rubber, nylon, vinyl or cross linked dextran. If cross-linked dextran is used, it is preferred that the molecular weight of the dextran polymer is between 50,000 and 500,000. The most preferable non-gellable polysaccharide is a non-gellable galactomannan macromolecule such a guar gum. A range of guar gum between approximately 0.01 kg to 100 kg, preferably between approximately 0.1 kg to 10 kg, and most preferably between approximately 0.5 kg to 2 kg is generally sufficient. Other non-gellable polysaccharides may include lucerne, fenugreek, honey locust bean gum, white clover bean gum and carob locust bean gum.

The matrix of the preferred embodiment preferably comprises polymerized chains of acrylamide monomer, wherein the acrylamide monomers are cross-linked with a cross-linking agent, a non-gellable polysaccharide, and an active agent or pharmaceutical directly encapsulated into micro-cavities therein. A range of acrylamide between approximately 1 kg to 100 kg, preferably between approximately 2 to 50 kg, and most preferably between approximately 5 kg to 20 kg is generally sufficient. A preferred matrix comprises a cross-linked polyacrylamide scaffolding that enmeshes guar gum as disclosed in U.S. Pat. No. 5,196,160 to Nangia, incorporated herein by reference.

The preservative agents incorporated into the matrices and devices of the present invention may be used for a variety of applications where there is a need for the presence of the active agent. A particularly preferred use is in the treatment and preservation of plant materials in both the agricultural and horticultural sectors.

The composition may also include additional antimicrobial agents, including but not limited to antifungal agents, antibacterial agents, anti-viral agents and anti-parasitic agents, growth factors, angiogenic factors, anaesthetics, mucopolysaccharides, and metals, disinfectants, antibiotics, cleaners, and other chemicals.

Examples of antimicrobial agents that can be used in the present invention include, but are not limited to, -8-hydroxyquinoline sulfate, 8-hydroxyquinoline citrate, aluminum sulfate, quaternary ammonium, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazuril, acyclovir, trifluorouridine, foscarnet, penicillin, gentamicin, ganciclovir, iatroconazole, miconazole, Zn-pyrithione, heavy metals including, but not limited to, gold, platinum, silver, zinc and copper, and their combined forms including, salts, such as chloride, bromide, iodide and periodate, and complexes with carriers, and other forms.

Multiple inactive ingredients may be optionally incorporated in the formulations. Examples of ingredients are emulsifiers, thickening agents, solvents, anti foaming agents, preservatives, fragrances, coloring agents, emollients, fillers, and the like.

An embodiment of the invention includes assaying plant material for the presence of a biofilm and/or determining one or more anti-biofilm agents effective against the biofilm. In accordance with the present invention, the effectiveness of a composition of the present invention may be determined by any process or means that involves contacting the biofilm with the active agent. An exemplary device and process is shown in U.S. Pat. Nos. 6,052,423 and 6,326,190, each incorporated herein by reference.

In the present invention, the plant material may be assayed using any assay device for detecting a biofilm and or for determining an anti-biofilm agent. An exemplary embodiment of the invention comprises determining one or more anti-biofilm agents, preferably using a biofilm assay device. One method includes, but is not limited to, analyzing biofilms and their reaction to anti-biofilm agents from exemplary device and process shown in U.S. Pat. Nos. 6,051,423; 6,051,423; 6,326,190; 6,410,256; 6,596,505; 6,599,696; and 6,599,714.

In the present invention, the preferred method is the Minimal Biofilm Eradication Concentration (MBEC) technique which consists of growing identical 24-hour biofilms on 96 pegs arrayed in 12 rows and 8 columns. The biofilms then are challenged with decreasing concentrations of selected antibiotics and/or biocides. After a certain challenge time (generally one hour), the biofilms are placed in 96 individual wells of growth media and ultra-sonicated for recovery of any surviving organisms. After culturing overnight, the wells are checked for turbidity. Clear, transparent wells indicate complete deactivation of the biofilm. Conversely, turbidity (“growth”) indicates lack of complete deactivation of the biofilm. Where needed, the bacterial suspensions can be cultured on agar plates, with or without 10-fold serial dilution, to estimate the surviving rate of the microorganisms in biofilms. Log reductions can be estimated to evaluate efficacy of biocide. In this method, the bacteria or fungi, after being incubated to form the biofilm, are treated with one or more anti-microbial agents. The biofilm may be analyzed using a device such as one of those listed in the previous paragraph.

The compositions and methods of the present invention may be used to treat biofilm in a wide range of environments and places. Treating biofilm, as used herein, refers to contacting a biofilm or similar structure with an anti-biofilm agent wherever biofilm may be found, expected to be found, or postulated to be found. One skilled in the art will readily recognize that the areas and industries for which the present invention is applicable is a vast number of processes, products, and places.

For example, the compositions and methods of the present invention are effective, or are expected to be effective, in preserving and/or disinfecting plant seeds. Exemplary seeds include, but are not limited to dry beans, pulse crops (e.g., peas, lentils, chickpeas, and faba beans), seeds from cereals, e.g., wheat and barley; and potato seeds.

In this aspect of the invention, the compositions and methods are suitable for treating against one or more microbial infections, including but not limited to diseases or conditions caused by Pseudomonas, Xanthomonas, C. flaccumfaciens, S. sclerotium, Pythium species, Fusarium species; H. solani; Streptomyces species, including scabies; Clavibacter species, Erwinia species; Listeria species, Campylobacter, species; Shigella species; and E. Coli.

Exemplary disease or conditions include, but are not limited to bacterial blight, brown spot, common blight, vascular wilt, white mold, root rots, head blight, silver scurf, dry rot, common scab, ring rot, and soft rot.

The compositions and methods of the present invention are also effective, or expected to be effective, in decontaminating, disinfecting, or protecting a wide assortment of surfaces. Exemplary surfaces include, but are not limited to agricultural surfaces, e.g., greenhouse, irrigation systems, storage facilities, and crates and bins; agricultural tools and equipment, including harvesting and processing equipment, conveyor belts, pickers, seeders, and cutters; food processing plants, centers, or equipment, including dairy plants, poultry plants, slaughter houses, seafood processing plants, fresh produce processing centers, and beverage processing centers.

The compositions and methods of the present invention are also effective, or expected to be effective, as a protective coating and/or as an ingredient in a protective coating. Exemplary areas include but are not limited to building, environmental, medical, dental, and industrial surfaces. Exemplary surfaces include but are not limited to hospitals, greenhouses, agricultural storage facilities, water systems, ships (e.g., biocorrosion), cables (e.g., biocorrosion), and pipelines (e.g., biocorrosion); and coatings themselves, e.g., paint, stain, and grout; medical devices, e.g., catheters and dialysis machines, or parts thereof; and dental implants and coatings.

The compositions and methods of the present invention are also effective, or expected to be effective, as a preservative for cosmetics, including but not limited to an ingredient of a cosmetic, or incorporated into the packaging of a cosmetic.

### DEFINITIONS

As used herein, plant material refers to any plant or vegetable, or parts thereof, including flowers, fruits, produce, seeds, stems, vessels, roots and wood fibers.

As used herein, anti-biofilm agent refers to any element, chemical, biochemical, or the like that is effective against a biofilm. Typical anti-biofilm agents are those that have anti-microbial, anti-bacterial, anti-fungal or anti-algal properties. Metal and metal compounds, preferably silver deposition compounds, have been shown generally to have anti-bacterial and ethylene inhibiting properties, and are preferred anti-biofilm agents in accordance with the present invention.

As used herein, preservatives or similar words include any element, chemical or biochemical or the like that can be used to preserve or extend the shelf like of a plant material, such as a cut flower. A preservative may be an anti-biofilm agent, or may be used in combination with an anti-biofilm agent, or may be used after an anti-biofilm agent is removed or degraded a biofilm.

“Sustained release” or “sustainable basis” are used to define release of atoms, molecules, ions or clusters of a noble metal that continues over time measured in hours or days, and thus distinguishes release of such metal species from the bulk metal, which release such species at a rate and concentration which is too low to be effective, and from highly soluble salts of noble metals such as silver nitrate, which releases silver ions virtually instantly, but not continuously, in contact with an alcohol or electrolyte.

MIC: Minimal Inhibitory Concentration—minimal concentration of an antimicrobial that is effective to inhibit microbial growth.

MBC: Minimal Bactericidal Concentration—minimal concentration of an antimicrobial that can effectively eradicate planktonic population of microorganisms.

MBEC: Minimal Biofilm Eradication Concentration—minimal concentration of an antimicrobial that can effectively eradicate biofilms.

pH: A measurement of the acidity of a solution. A pH of 7.0 is neutral. Lower pH values are more acidic and above 7.0 they are alkaline.

Planktonic: Microorganisms growing as floating, single cells, which is part of their life cycle.

Post-harvest: The period from harvest on for crops such as cut flowers and potted plants, and vegetables.

Shattering: Falling off flowers and/or petals.

Sustained release: release of atoms, molecules, ions or clusters of an antimicrobial or noble metal that continues over time, measured in hours or days.

Vaselife: The life of flowers starting when they arrive at the final consumer.

### EXAMPLES

The present invention will be further described in detail with reference to the following working examples. Note, however, that the present invention is not restricted to these examples.

### Example 1

### Oxysilver Powder Anti-Microbial Activity Against Erwinia carotovora Subsp. carotovora (Ecc), the Soft Rot of Vegetables Pathogen, in Comparison to Nanocrystalline Silver Powder

TABLE 1
Ecc biofilm susceptibility to Oxysilver and nanocrystalline powder
Ag30 and Ag 100 (Nanotechnologies, Inc.) at 24 h contact time.

| | Ag30 | Ag100 | Oxy |
|---|---|---|---|
| 500 ppm | 0 | 0 | 0 |
| | 0 | 0 | 0 |
| | 0 | 0 | 0 |
| | 0.00 | 0.00 | 0.00 |
| 200 ppm | 0 | 0 | 0 |
| | 0 | 0 | 0 |
| | 0 | 0 | 2.11 |
| | 0.00 | 0.00 | 0.70 |
| 100 ppm | 0 | 0 | 0 |
| | 0 | 0 | 0 |
| | 0 | 0 | 1.95 |
| | 0.00 | 0.00 | 0.65 |
| 50 ppm | 0 | 1.30 | 1.60 |
| | 1.60 | 0.00 | 1.00 |
| | 1.00 | 1.48 | 2.00 |
| | 0.87 | 0.93 | 1.53 |
| 0 ppm | 3.85 | 3.70 | 3.48 |
| | 3.78 | 3.60 | 3.90 |
| | 3.60 | 3.60 | 3.90 |
| | 3.74 | 3.63 | 3.76 |

Cell counts expressed in log10, silver compound concentration in parts per million.

TABLE 2
Log reduction of Ecc biofilms treated with Oxysilver and nanocrystalline
powder Ag30 and Ag 100 at 24 h contact time.

| | Ag30 | Ag100 | Oxy1 |
|---|---|---|---|
| 500 ppm | 3.74 | 3.63 | 3.76 |
| 200 ppm | 3.74 | 3.63 | 3.06 |
| 100 ppm | 3.74 | 3.63 | 3.11 |
| 50 ppm | 2.87 | 2.7 | 2.23 |

### Conclusion

Oxysilver was as efficacious as nanocrystalline silver as anti-microbial against plant pathogenic Erwinias.

### Example 2

### Oxysilver Powder Anti-Microbial Activity Against Both Biofilm and Planktonics of Pseudomonas syringae Pv. phaseolicola Hb-9, a Bean Halo Blight Pathogen, in Comparison to Other Seed Treatment Products Such as Copper Sulfate

TABLE 3
Pseudomonas syringae pv. phaseoticola HB-
9 biofilm and planktonic susceptibility to Oxysilver
and copper based seed treatment products.

| | Bacteria/ | Bacteria/biofilm |
|---|---|---|
| Compound/ | growth | growth |
| concentration | PspHB-9 Planktonic | PspHB-9 Biofilm |
| Oxysilver | | |
| 100 ppm | 0 | 0 |
| 500 ppm | 0 | 0 |
| 1000 ppm | 0 | 0 |
| Copper sulfate | | |
| 1250 ppm | 2.95 | 5.15 |
| 2900 ppm | 0 | 0 |
| 4600 ppm | 0 | 0 |
| H₂0 (0 ppm) | | |
| | 5.66 | 6.05 |
| | 5.75 | 5.97 |
| | 5.75 | 6.67 |
| | 5.72 | 6.23 |

Cell counts expressed in log10, silver compound concentration in parts per million.

TABLE 4
Log reduction of Pseudomonas syringae pv. phaseolicola planktonic
and biofilms treated with Oxysilver and copper sulfate for 2 h.

| | | Bacteria/ | Bacteria/biofilm |
|---|---|---|---|
| Compound/ | | growth | growth |
| concentration | | PspHB-9 Planktonic | PspHB-9 Biofilm |
| Oxysilver | | | |
| 100 | ppm | 5.72 | 6.23 |
| 500 | ppm | 5.72 | 6.23 |
| 1000 | ppm | 5.72 | 6.23 |
| Copper sulfate | | | |
| 5,000 | ppm | 2.77 | 1.08 |
| 11,460 | ppm | 5.72 | 6.23 |
| 18,300 | ppm | 5.72 | 6.23 |

| H₂0 (0 ppm) | | |
|---|---|---|
| | 5.66 | 6.05 |
| | 5.75 | 5.97 |
| | 5.75 | 6.67 |
| | 5.72 | 6.23 |

### Conclusions

Oxysilver led to 100% eradication of planktonic and biofilm at the lowest concentration (50× lower than the lowest copper concentration tested, which did cause reduction in biofilms)

Copper sulfate: ˜48% reduction of planktonics and 17% reduction of biofilms at lowest concentration (5000 ppm). Needed at least 11460 ppm for it to be effective.

Oxysilver is at least >50 times to 114× more effective in killing this microorganisms than copper sulfate.

### Example 3

### Evaluation of Oxysilver on Dry Bean Seed Germination and Emergence—Preliminary Toxicity Analysis Conducted in Greenhouse

Germination was measured directly for 50 seeds from each treatment at 5- and 10-days after plating on solid agar media. Germination was also measured indirectly for each treatment as emergence of seedlings from 20 seeds sown five/pot in four pots in the greenhouse. The results for germination on plates are shown in Tables 6-. Statistical comparisons of germination data using single variable ANOVA reveals that there are no significant differences in germination of seeds from any of the treatments. This result indicates that the treatments applied to dry bean seeds, including the experimental MBEC products, have no significant effect on seed germination.

TABLE 5
Products and compounds tested

| Product | Formulation | Rate |
|---|---|---|
| Negative Control #1 | Sterile Water | 5-mL or 1-kg per 1-kg seed |
| Negative Control #1 | Talc powder | 5-g to 1-kg seed |
| Ag Streptomycin (positive | 62.6% streptomycin sulphate; 50% | 5 mL of 1% solution to 1 kg seed |
| standard) | streptomycin base | |
| MBEC #2A-wet | Oxysilver salts in water | 5-mL of 0.05% solution to 1 kg seed |
| MBEC #2B-wet | Oxysilver salts in water | 5-mL of 0.1% solution to 1 kg seed |
| MBEC #2A- dry | Oxysilver salts in talc powder | 5-g of 0.05% talc mixture to 1 kg seed |
| MBEC #2B- dry | Oxysilver salts in talc powder | 5-g of 0.1% talc mixture to 1 kg seed |

TABLE 6
Germination of dry bean seeds cultivar Othelo on King's B agar plates.

| | Sterile | Talc | Ag-Strep | Oxysilver | Oxysilver | Oxysilver | Oxysilver |
|---|---|---|---|---|---|---|---|
| | Water | Powder | 1% | 0.05% in water | 0.1% in water | 0.05% in talc | 0.1% in talc |
| Plate 1 | 5 | 4 | 5 | 5 | 5 | 5 | 4 |
| Plate 2 | 5 | 5 | 4 | 5 | 5 | 5 | 5 |
| Plate 3 | 5 | 5 | 5 | 5 | 5 | 5 | 5 |
| Plate 4 | 5 | 5 | 5 | 5 | 5 | 5 | 5 |
| Plate 5 | 5 | 5 | 5 | 5 | 5 | 5 | 4 |
| Plate 6 | 4 | 5 | 5 | 5 | 5 | 5 | 5 |
| Plate 7 | 5 | 5 | 4 | 5 | 5 | 5 | 5 |
| Plate 8 | 5 | 5 | 5 | 5 | 5 | 5 | 5 |
| Plate 9 | 5 | 5 | 4 | 5 | 5 | 5 | 5 |
| Plate 10 | 5 | 5 | 4 | 5 | 5 | 5 | 5 |
| SUM | 49 | 49 | 46 | 50 | 50 | 50 | 48 |
| MEAN | 4.9 | 4.9 | 4.6 | 5 | 5 | 5 | 4.8 |

TABLE 7
Germination of dry bean seeds cultivar AC-POLARIS on King's B agar plates.

| | Sterile | Talc | | Oxysilver 0.05% | Oxysilver | Oxysilver | Oxysilver |
|---|---|---|---|---|---|---|---|
| | Water | Powder | Ag-Strep 1% | in water | 0.1% in water | 0.05% in talc | 0.1% in talc |
| Plate 1 | 5 | 3 | 5 | 5 | 4 | 5 | 5 |
| Plate 2 | 4 | 3 | 4 | 3 | 5 | 5 | 5 |
| Plate 3 | 4 | 4 | 3 | 5 | 3 | 5 | 5 |
| Plate 4 | 4 | 4 | 3 | 4 | 1 | 4 | 5 |
| Plate 5 | 4 | 5 | 5 | 2 | 4 | 5 | 4 |
| Plate 6 | 4 | 4 | 4 | 3 | 4 | 5 | 4 |
| Plate 7 | 3 | 3 | 4 | 4 | 3 | 5 | 5 |
| Plate 8 | 3 | 3 | 4 | 5 | 5 | 5 | 4 |
| Plate 9 | 4 | 3 | 3 | 4 | 4 | 4 | 5 |
| Plate 10 | 5 | 3 | 3 | 3 | 5 | 3 | 4 |
| SUM | 40 | 35 | 38 | 38 | 38 | 46 | 46 |
| MEAN | 4 | 3.5 | 3.8 | 3.8 | 3.8 | 4.6 | 4.6 |

### Example 4

### Emergence of Seedlings in the Greenhouse

TABLE 8
Emergence of seedling of ‘Othello’ (pinto beans) treated with silver in preliminary
greenhouse trials, three weeks after planting.

| | | | | | | | |
|---|---|---|---|---|---|---|---|
| | | | | Oxysilver 0.05% | Oxysilver | Oxysilver | Oxysilver |
| Replication | Sterile Water | Talc Powder | Ag-Strep 1% | in water | 0.1% in water | 0.05% in talc | 0.1% in talc |

Experiment 1-Emergence Data for ‘Othello’ Greenhouse Trial #1

| | | | | | | | |
|---|---|---|---|---|---|---|---|
| 1 | 3 | 4 | 5 | 5 | 4 | 4 | 4 |
| 2 | 3 | 5 | 3 | 4 | 4 | 4 | 3 |
| 3 | 5 | 4 | 3 | 5 | 4 | 5 | 5 |
| 4 | 5 | 5 | 3 | 5 | 3 | 5 | 4 |
| SUM | 16 | 18 | 14 | 19 | 15 | 18 | 16 |
| MEAN | 4 | 4.5 | 3.5 | 4.75 | 3.75 | 4.5 | 4 |

Experiment 2-Emergence Data for ‘Othello’ Greenhouse Trial #2

| | | | | | | | |
|---|---|---|---|---|---|---|---|
| 1 | 4 | 4 | 5 | 5 | 4 | 5 | 4 |
| 2 | 5 | 3 | 4 | 5 | 5 | 4 | 4 |
| 3 | 5 | 4 | 5 | 5 | 5 | 5 | 5 |
| 4 | 4 | 3 | 5 | 5 | 5 | 5 | 4 |
| SUM | 18 | 14 | 19 | 20 | 19 | 19 | 17 |
| MEAN | 4.5 | 3.5 | 4.75 | 5 | 4.75 | 4.75 | 4.25 |

Total and Mean of Trials 1 and 2 for Othelo seed emergence

| | | | | | | | |
|---|---|---|---|---|---|---|---|
| TOTAL | 34 | 32 | 33 | 39 | 34 | 37 | 33 |
| MEAN | 4.25 | 4 | 4.125 | 4.875 | 4.25 | 4.625 | 4.125 |

TABLE 9
Emergence of seedling of ‘AC Polaris’ (white beans) treated with silver in preliminary
greenhouse trials three weeks after planting.
Experiment 3-Emergence Data for ‘AC Polaris’ Greenhouse Trial #1

| | | | | Oxysilver | Oxysilver | Oxysilver | Oxysilver |
|---|---|---|---|---|---|---|---|
| Replication | Sterile Water | Talc Powder | Ag-Strep 1% | 05% in water | 0.1% in water | 0.05% in talc | 0.1% in talc |
| 1 | 5 | 4 | 5 | 4 | 3 | 4 | 5 |
| 2 | 4 | 5 | 2 | 4 | 4 | 4 | 5 |
| 3 | 3 | 5 | 4 | 3 | 4 | 5 | 5 |
| 4 | 2 | 5 | 2 | 4 | 5 | 5 | 5 |
| SUM | 14 | 19 | 13 | 15 | 16 | 18 | 20 |
| MEAN | 3.5 | 4.75 | 3.25 | 3.75 | 4 | 4.5 | 5 |

### Example 5

### Microbial Recovery from Othelo and AC Polaris Seeds Treated, with Oxysilver in Comparison to Untreated Seeds

TABLE 10
Experiments description.

| Experiment | Objective |
|---|---|
| 1. Microbial recovery | To determine the type and number of |
| from non-inoculated, | naturally occurring microorganisms that can |
| non-sterile seeds | be removed from the bean seed coat and |
| | cultured on agar medium. |
| 2. Microbial recovery | To determine the efficacy of oxysilver as a |
| from non-sterilized, | bean seed treatment, by measuring |
| non-inoculated seeds | eradication naturally occurring |
| treated with | microorganisms from the seed surface. |
| oxysilver | |
| 3. Microbial recovery | Same as Experiment #1 except that seeds |
| from non-sterilized | are artificially infested with the halo blight |
| seeds inoculated with | pathogen, Pseudomonas syringae pv. |
| | phaseolicola. |
| 4. Microbial recovery | Same as Experiment #2 except that seeds |
| from non-sterilized | are artificially infested with the halo blight |
| seeds inoculated | pathogen, Pseudomonas syringae pv. |
| with Pseudomonas | phaseolicola.. |
| syringae pv. | |
| phaseolicola treated | |
| with oxysilver | |

Dry Bean Seed Cultivars Used in these Experiments: Sample Description

1. Othelo—Pinto beans—These seeds were collected straight from the field, including seeds that would normally be discarded during processing in a commercial seed treatment facility—damaged, discoloured, wilted or small seeds. These seeds were not cleaned either, which means these seeds still contain soil, dirt, plant debris, etc. Therefore, this seed lot was expected to provide a good model for a high natural microbial challenge, with good diversity of organisms and high microbial recovery expected. These seeds were overall more challenging to treat, generating more variable results as expected for such minimally processed seeds.

2. AC Polaris—White beans—These seeds were processed and cleaned in a commercial seed treatment facility. Processing leads to rejection of small, damaged, and/or discoulored seeds, leading to healthier seed lots. After processing, seeds were washed and cleaned, resulting in a seed lot with no soil, dirt or debris attached to it. Due to these pre-treatments, lower diversity and numbers of microorganisms are expected to be recovered from these seeds, especially in comparison to the highly challenging lot of Othelo seeds used in this study. Good model for testing artificially inoculated seeds.

### Scoring of Seed Microbial Contamination

Experiments were conducted by treating lots of Othelo and AC Polaris seeds with challenges described in Table 10. Seeds were placed individually onto wells of 12-well microtiter plates and treated for 1 h with silver and/or water. After treatment, seeds were rinsed by dipping into fresh phosphate buffer, transferred to a new 12-well microtiter plate containing fresh phosphate buffer and sonicated for 15-30 min. to allow for surviving bacterial and fungal to be recovered into the liquid. After 6 days, the wells of 12-well plates containing the seeds were scored based on both:
the turbidity of the liquid in which they were originally immersed for treatment, on day 6.
seed surface area covered by bacterial and/or fungal colonization, also on day 6.

Scores varied from:
(0)—No microorganisms were observed on seed surfaces and no turbidity was evident after 6 days
(1)—Minimal turbidity and/or no more than 20% of the external area of the seed visibly covered by microorganisms
(2)—More than 20% but less than 50% of seed surface covered by microorganisms and/or turbidity above that observed on (1) but far from matching a McFarland turbidity standard scale of 0.5.
(3)—At least 50% of seed surface colonized by microorganisms and heavier turbidity approaching the turbidity in the lowest standard of McFarland scale—0.5.
(4)—More than 50% of seed surface colonized by bacteria and/or fungi and/or heavy turbidity (bottom of well of 12-well plate barely visible)
(5)—Seed surface completely covered by microorganisms and/or maximum turbidity, “milky” looking liquid cultures.

Six seeds were scored in each experiment, which have been repeated 3 times independently. Numbers presented on Tables 11, 13 and 15 represent the mean of 6 scores. Efficacy of oxysilver in reducing the microbial populations on seeds was measured based on overall scores for bacteria, fungi and total microbial scores (bacteria and fungi together) and are represented as percentage of killing on Tables 12, 14 and 16.

### Experiment 1

Othelo seeds: Four different types of fungi were recovered from Othelo seeds, mostly on non-inoculated treatments, based on preliminary visual identification, including one with morphology and spore coloration indicative of Fusarium sp. Five to six types of bacterial colonies were also recovered from non-inoculated Othelo seeds. When seeds were inoculated with the halo blight pathogen, fungal occurrence and numbers were reduced, which is not surprising due to the broad spectrum of antibiotics produced by Pseudomonads in general, which usually outcompete other microorganisms when colonizing surfaces.

AC Polaris seeds: AC Polaris seeds have been pre-cleaned in a commercial seed cleaner facility, ans recovery data confirmed our expectation of lower microbial recovery from these seeds on natural, non-inoculated seeds. Interestingly, when these seeds were inoculated with Pseudomodas, probably due to the diminished amount of competitors on the surface to be colonized, bacterial counts were really high on untreated seeds.

Data is summarized on tables 11 and 12 below.

TABLE 11
Estimation of bacterial and fungal populations recovered from
untreated Othelo and AC-Polaris seed surfaces in comparison to
oxysilver-treated seed surfaces, based on a scoring system.

| | | | | Total microbial |
|---|---|---|---|---|
| Seed variety | | Bacteria | Fungi | score |
| | Non-inoculated | | | |
| Othelo | H₂0 | 4.0 | 3.83 | 7.83 |
| Othelo | Oxysilver (0.1%) | 0.83 | 1.16 | 1.99 |
| AC Polaris | H₂0 | 2.0 | 1.5 | 3.5 |
| AC Polaris | Oxysilver (0.1%) | 0.66 | 0 | 0.66 |
| | Inoculated w/ | | | |
| | bacteria | | | |
| Othelo | H₂0 | 3.66 | 1.5 | 5.16 |
| Othelo | Oxysilver 1 (0.1%) | 1.5 | 0.33 | 1.83 |
| AC Polaris | H₂0 | 5.0 | 0 | 5 |
| AC Polaris | Oxysilver 1 (0.1%) | 0 | 0 | 0 |

TABLE 12
Efficacy of oxysilver compound in reducing microbial infestation
of seeds, showing estimated reduction of bacterial and fungal
population, in addition to overall microbial load (bacteria and
fungi) as a percentage of that observed on untreated seeds.

| | Bacterial | Fungal | Total microbial |
|---|---|---|---|
| Oxysilver Treatment | reduction | reduction | reduction |
| Othelo Non-inoculated | 80% | 70% | 75% |
| AC Polaris Non-inoculated | 67% | 100% | 81% |
| Othelo Inoculated | 41% | 78% | 65% |
| AC Polaris Inoculated | 100% | Non applicable | 100% |

### Experiment 2

The same trend observed in Experiment 1 happened in experiment 2.

Othelo seeds: Three different types of fungi were recovered from Othelo seeds, mostly on non-inoculated treatments, based on preliminary visual identification. Five types of bacterial colonies were recovered from non-inoculated Othelo seeds. When seeds were inoculated with the halo blight pathogen, fungal occurrence reduced, and organisms recovered were comprised mostly of the inoculated pseudomonad.

AC Polaris seeds: Again recovery data showed lower microbial recovery from these seeds on natural, non-inoculated seeds. When inoculated with Pseudomonads, bacterial counts were high on untreated seeds.

Data is summarized on Table 13 and 14.

TABLE 13
Estimation of bacterial and fungal populations recovered from
untreated Othelo and AC-Polaris seed surfaces in comparison to
oxysilver-treated seed surfaces, based on a scoring system.

| | | | | Total microbial |
|---|---|---|---|---|
| Seed variety | | Bacteria | Fungi | score |
| | Non-inoculated | | | |
| Othelo | H₂0 | 2.16 | 3.66 | 5.82 |
| Othelo | Oxysilver (0.1%) | 0.83 | 0 | 0.83 |
| AC Polaris | H₂0 | 1.66 | 1.33 | 2.99 |
| AC Polaris | Oxysilver (0.1%) | 0.16 | 0.5 | 0.66 |
| | Inoculated w/ | | | |
| | bacteria | | | |
| Othelo | H₂0 | 3.00 | 2.66 | 5.66 |
| Othelo | Oxysilver 1 (0.1%) | 0.66 | 0.66 | 1.32 |
| AC Polaris | H₂0 | 4.16 | 0 | 4.16 |
| AC Polaris | Oxysilver 1 (0.1%) | 0.16 | 0 | 0.16 |

TABLE 14
Efficacy of oxysilver compound in reducing microbial infestation
of seeds, showing estimated reduction of bacterial and fungal
population, in addition to overall microbial load (bacteria and
fungi) as a percentage of that observed on untreated seeds.

| | Bacterial | Fungal | Total microbial |
|---|---|---|---|
| Oxysilver Treatment | reduction | reduction | reduction |
| Othelo Non-inoculated | 62% | 100% | 86% |
| AC Polaris Non-inoculated | 90% | 62% | 78% |
| Othelo Inoculated | 78% | 75% | 77% |
| AC Polaris Inoculated | 96% | Non applicable | 96% |

### Experiment 3

Diversity data was similar to previous experiments.

TABLE 15
Estimation of bacterial and fungal populations recovered from
untreated Othelo and AC-Polaris seed surfaces in comparison to
oxysilver-treated seed surfaces, based on a scoring system.

| | | | | Total microbial |
|---|---|---|---|---|
| Seed variety | Non-inoculated | Bacteria | Fungi | score |
| Othelo | H₂0 | 2.83 | 3.16 | 5.99 |
| Othelo | Oxysilver (0.1%) | 0.33 | 0.83 | 1.16 |
| AC Polaris | H₂0 | 2.0 | 1.16 | 3.16 |
| AC Polaris | Oxysilver (0.1%) | 0 | 0 | 0 |
| Othelo | H₂0 | 4.16 | 3.66 | 7.82 |
| Othelo | Oxysilver (0.1%) | 1.66 | 0 | 1.66 |
| AC Polaris | H₂0 | 4.16 | 0 | 4.16 |
| AC Polaris | Oxysilver (0.1%) | 0 | 0 | 0 |

TABLE 16
Efficacy of oxysilver compound in reducing microbial infestation
of seeds, showing estimated reduction of bacterial and fungal
population, in addition to overall microbial load (bacteria and
fungi) as a percentage of that observed on untreated seeds.

| | Bacterial | Fungal | Total microbial |
|---|---|---|---|
| Oxysilver Treatment | reduction | reduction | reduction |
| Othelo Non-inoculated | 88% | 74% | 81% |
| AC Polaris Non-inoculated | 100% | 100% | 100% |
| Othelo Inoculated | 60% | 100% | 79% |
| AC Polaris Inoculated | 100% | Non applicable | 100% |

### Conclusions:

Oxysilver showed good bactericide and fungicide efficacy against various bacterial and fungal species associated with seed surfaces. The data was consistent through 3 independent replications of this experiment.

These results, in combination with the results summarized by other examples presented in this document, demonstrate the potential of oxysilver as a unique seed treatment product. Products currently available are usually either bactericidal of fungicidal, not both.

### REFERENCES CITED

1. Van Doorn, W. G., De Witte, Y. and Perik, R. J. J. 1990. Effect of anti-microbial compounds on the number of bacteria in stems of cut rose flowers. J. Appl. Bacteriol. 68:117-122.
2. Jones, R. B. and Hill, D. M. 1993. The effect of germicides on the longevity of cut flowers. J. Am. Soc. Hortic. Sci. 118: 350-354.
3. Knee, M. 2000. Selection of biocides for use in floral preservatives. Postharvest Biol. Technol. 18: 227-234.
4. Ichimura, K., Kojima, K., and Goto, R. 1999. Effects of temperature, 8-hydroxyquinoline sulphate and sucrose on the vase life of cut rose flowers. Postharvest Biol. Technol. 15: 33-40.
5. Mayak, S., Garibaldi, E. A., and Kofranek, A. M. 1977. Carnation flower longevity: microbial populations as related to silver nitrate impregnation. J. Am. Soc. Hortic. Sci. 102: 637-639.
6. Marousky, F. J. 1981. Inhibition of cut flower bacteria by 8-hydroxyquinoline citrate. Acta Hort. 113:81-88.
7. Van Doorn, W. G. and Perik, R. R. J. 1990. Hydroxyquinoline citrate and low pH prevent vascular blockage in stems of cut rose flowers by reducing the number of bacteria. J. Am. Soc. Hortic. Sci. 115(6):979-981.
8. Liao, L. J., Lin, Y. H., Chen, W. S., and Cheng, Y. M. 2000. Postharvest life of cut rose flowers as affected by silver thiosulfate and sucrose. Bot. Bull. Acad. Sin. 41: 299-303.
9. Parups, E. V., and Peterson, E. A. 1973. Inhibition of ethylene production in plant tissues by 8-Hydroxyquinoline. Can. J. Plant Sci. 53: 351-353.
10. Futaki, K., Shigeno; K., and Hoshi, K. 1996. Preservative of cut flowers U.S. Pat. No. 5,536,155
11. Clerkx, A. C. M., Boekestein, A. and Put, H. M. C. 1988. Scanning electron microscopy of the stem cut flowers of rosa cv. ‘Sonia’ and gerbera cv. ‘fleur’. Acta Hort. 261:97-105.
12. Van Doorn, W. G. 1999. Vascular occlusion in cut flowers. I. General principles and recent advances. Acta Hort. 482: 59-63
13. Put, H. M. C. and Conway, C. C. 1986. Investigations into the influence of the microflora from stems of cut flower on the vase-life of rose ‘Sonia’, gerbera ‘fleur’ and chrysanthemum ‘spider’. Acta Hort. 181:415-418.
14. Stoodley. P., Sauer, K., Davies, D. G., and Costerton, J. W. 2002. Biofilms as complex differentiated communities. Annu Rev Microbiol 56:187-209.
15. Marques, L. L. R., Ceri, H., Manfio, G. P., Reid, D. M., and Olson, M. E. 2002. Characterization of biofilm formation by Xylella fastidiosa in vitro. Plant Dis. 86: 633-638.
16. Marques, L. L. R., De Boer, S. H., Ceri, H. and Olson, M. E. 2003. Evaluation of biofilms formed by Clavibacter michiganensis subsp. sepedonicus. Phytopathology 93:S57.
17. De Boer, S. H., Marques, L. L. R., and Olson, M. E. 2004. Biofilms: Potential for Trouble. Potato Storage International 1: 28-31.
18. Van Doorn, W. G., De Stitger, H. C. M., De Witte, Y., and Boekestein, A. 1991. Microorganisms at the cut surface and in the xylem vessels of rose stems: a scanning electron microscope study. J. Appl. Bacteriol. 70:34-39.
19. Burrell, R. E., Apte, P. S., Gill, K. S., Pretch, R. J., and Morris, L. R. 1999. Process for producing anti-microbial effect with complex silver ions. U.S. Pat. No. 5,985,308.
20. Bromberg, L. E., Spacciapoli, P., Rothstein, D. M., Braman, V. M. Buxton, D. K., Tonetti, M. S., and Friden, P. M. 2000. Silver ion as antimicrobial agent for sustained subgingival delivery. J. Dent. Res. 79: 575.
21. Alt, V., Bechert, T., Steinrucke, P., Wagener, M., Seidel, P., Dingeldein, E., Domann, E., Schnettler, R. 2004. An in vitro assessment of the antibacterial properties and cytotoxicity of nanoparticulate silver bone cement. Biomaterials 25: 4383-4391.
22. Yin, HQ, Langford, R, and Burrell, RE 1999. Comparative evaluation of the antimicrobial activity of ACTICOAT* Antimicrobial Barrier Dressing. J. Burn Care Rehabil. 20: 195-200.
23. Sondi, I., and Salopek-Sondi, B. 2004. Silver nanoparticles as antimicrobial agent: a case study on E-coli asa model for Gram-negative bacteria. J. Colloid Interface Sci. 275:177-182.
24. Wright J. B., Lam, K., Buret, A. G., Olson, M. E. and Burrell, R. E. 2002. Early healing events in a porcine model of contaminated wounds: effects of nanocrystalline silver on matrix metalloproteinases, cell apoptosis, and healing. Wound Repair. Regen. 10:141-151.
25. Wright, J. B., Lam, K., Hansen, D. and Burrell, R. E. 1999. Efficacy of topical silver against fungal burn wound pathogens. Am. J. Inf. Control 27: 344-350 (1999)
26. Bromberg, L. E., Braman, V. M., Rothstein, D. M., Spacciapoli, P., O'Connor, S. M. Nelson, E. J., Buxton, D. K., Tonetti, M. S., Friden, P. M. 2000. Sustained release of silver from periodontal wafers for treatment of periodontitis. J. Control. Release 68: 63-72.
27. National Advisory Committee on Microbiological Criteria for Foods. 1999. Microbiological safety evaluations and recommendations on sprouted seeds. Int. J. Food Microbiol. 52:123-153
28. Taormina, P. J., Beuchat, L. R., and Slutsker, L. 1999. Infections associated with eating seed sprouts: An international concern. Emerg. Infect. Dis. 5:626-634.

Although a few preferred embodiments have been described, it will be appreciated by those skilled in the art that various changes and modifications might be made without departing from the scope of the invention. The terms and expressions in the preceding specification have been used therein as terms of description and not of limitation, and there is not intention in the use of such terms and expressions of excluding equivalents of the features shown and described or portions thereof, it being recognized as the scope of the invention as defined and limited only by the claims that follow.

We claim:

1. A method for treating plant material comprising contacting a plant material or a portion thereof with a composition that comprises at least one preservative agent, said preservative agent comprising a silver deposition compound, thereby preserving the plant material.

2. The method claim 1 wherein said preservative agent is a silver deposition compound comprising:
a aqueous solution of silver nitrate,
an oxidizing agent, preferably potassium persulfate, wherein the amount of oxidizing agent is selected to be a stoichiometrically appropriate amount relative to the amount of the ions of the metal; and
anions comprised of at least one acid

3. The method of claim 1 wherein contacting a plant or portion thereof includes contacting a seed, root, stem, leaf, flower, and combinations thereof.

4. The method of claim 1 wherein preserving the plant material comprises treating the plant material against one or more biofilms.

5. The method of claim 4 wherein treating the plant material against one or more biofilms comprises eradicating or reducing the biofilm.

6. The method of claim 4 wherein treating the plant material against one or more biofilms comprises treating the plant material against one or more species selected from the group consisting of Erwinia species, Pseudomonas species, Fusarium species, and variants thereof.

7. A method of treating biofilm comprising contacting the biofilm with an anti-biofilm agent, wherein said anti-biofilm agent comprises a silver deposition compound.

8. The method of claim 7 wherein said anti-biofilm agent comprises a high valent silver ion, and mixtures thereof.

9. The method of claim 7 wherein the biofilm is located on or in a plant or portion thereof.

10. The method of claim 7 wherein the biofilm is located on or in a medical device or portion thereof.

11. The method of claim 7 further comprising at least one additional anti-biofilm agent.

12. The method of claim 7 wherein said anti-biofilm agent further comprises treating planktonic bacteria.

13. An anti-biofilm composition comprising a silver deposition compound.
